(19) United States
(12) Patent Application Publication
(10) Pub. No.: US 2026/0157707 A1
(43) Pub. Date: Jun. 11, 2026

(54) SYSTEM AND METHOD FOR DISPLAYING IMPEDANCE INFORMATION FOR PHYSIOLOGIC SENSORS

(71) Applicant: Drägerwerk AG & Co. KGaA, Lübeck (DE)

(72) Inventors: Lianna Colombo, Phoenix, AZ (US); Devin Weidner, Litchfield, NH (US); Isabelle Schacht, Berlin (DE); Karolin Lüneburg, Berlin (DE)

(73) Assignee: Drägerwerk AG & Co. KGaA, Lübeck (DE)

(21) Appl. No.: 18/707,286

(22) PCT Filed: Nov. 3, 2023
(86) PCT No.: PCT/IB2022/060610

§ 371 Date: May 3, 2024

Related U.S. Application Data
(60) Provisional application No. 63/275,220, filed on Nov. 3, 2021.

(51) Int. Cl. A61B 5/00; A61B 5/339; A61B 5/369

(52) U.S. Cl. CPC A61B 5/743; A61B 5/339; A61B 5/369

(57) ABSTRACT

A system and method for displaying, on a display that is part of a patient monitoring system, electrode-level impedance status and electrode location for a physiologic sensor. The electrode location may be superimposed on a representation of all or a portion of human body. Additional warning information is provided on the display if an electrode has an impedance status other than low impedance.

### BACKGROUND

In a health care environment, electrodes placed on the patient's skin are used as part of a variety of sensors for the purpose of gathering patient physiologic data (“electrode-based patient sensors”). Examples of the types of electrode-based patient sensors include electrocardiogram (“ECG”) sensors, neuromuscular transmission (“NMT”) sensors, electroencephalogram (“EEG”) sensors, and brain monitoring external (“BISx”) sensors. Each of these types of sensors use multiple electrodes.

An electrode for a physiologic sensor can become unable to provide an accurate physiologic measurement for a number of reasons. For example, there may be inadequate contact between the patient's skin and the electrode pad, a lack of sufficient conductive gel, insufficient pressure being applied during application of the electrode, or an adhesive failure (from excess skin moisture or patient movement). Other possible causes include, for example, poor electrical contact between a wire lead and the electrode pad and poor electrical contact between the wire lead and the sensor housing.

Identifying a problematic electrode can be difficult for clinicians, particularly for sensors having a significant number of electrodes. The diagnostic process is time consuming and error-prone. Moreover, avoiding delays or gaps in the display of accurate physiologic data can be important for patient care—such as ECG data for a patient experiencing a heart attack. Therefore, there is a need for a system that enables clinicians to quickly and efficiently identify the existence of a problematic electrode and to locate that electrode on the patient's body.

### SUMMARY

In one exemplary embodiment, electrode data associated several types of electrode-based sensor is stored in patient monitoring system. The electrode data includes, for each electrode, a label, a location on a representation of at least a portion of a human body or an electrode-retaining device, and values for a plurality of impedance statuses, including a low impedance status, a high impedance status, and, optionally, a medium impedance status. When a sensor type is selected, a graphical representation of the location for each of the plurality of electrodes for that sensor type is displayed, along with an impedance status for each of the plurality of electrodes.

A purpose of the embodiments disclosed herein is to provide a clinician with a consistent GUI experience across multiple types of electrode-based sensors. For each sensor, the location of each electrode on the human body is graphically shown in a location window. When an impedance tab is selected, impedance information for each electrode is provided in a control window. When the impedance status of at least one electrode is outside of ideal specifications, additional warnings may be provided in other parts of the GUI for the purpose of alerting the clinician.

### BRIEF DESCRIPTION OF THE DRAWINGS

For a more complete understanding of the present invention, reference is made to the following detailed description of embodiments considered in conjunction with the accompanying drawings, in which:

FIG. 1 is a schematic diagram of an exemplary physiological monitoring system with a plurality of surface ECG leads connected to a patient;

FIG. 2 is an exemplary graphical user interface for an electrode-based sensor, showing chest view impedance status information for a six-lead ECG sensor with all electrodes being at low impedance status;

FIG. 3 is the graphical user interface of FIG. 2, displaying some electrodes having medium and high impedance status and one medium impedance electrode selected for visual salience;

FIG. 4 is the graphical user interface of FIG. 2, displaying placement of the electrodes instead of electrode impedance status information;

FIG. 5 is a flow chart showing the steps and actions associated with a user selection of an impedance view;

FIG. 6 is a flow chart showing the steps and actions associated with a user selection of a placement view;

FIG. 7 is a flow chart showing the steps associated with detection and display of electrode impedance status;

FIG. 8 is the graphical user interface of FIG. 2, showing a twelve-lead ECG sensor, displaying electrodes having medium and high impedance and a fully body graphical representation;

FIG. 9 is the graphical user interface of FIG. 2, showing a twelve-lead ECG sensor, displaying electrodes having medium and high impedance and a chest view graphical representation;

FIG. 10 is the graphical user interface of FIG. 2, showing a twelve-lead ECG sensor, displaying electrodes having medium and high impedance and a limbs only graphical representation;

FIG. 11 is the graphical user interface of FIG. 2 (chest view), displaying some electrodes having lead off and electrode off status;

FIG. 12 is the graphical user interface of FIG. 8 (full body view), displaying some electrodes having lead off and electrode off status;

FIG. 13 is an exemplary graphical user interface for a BISx sensor, showing impedance status information and the location of the sensor electrodes superimposed on the graphical representation of an electrode retaining device;

FIG. 14 is the graphical user interface of FIG. 13, showing the location of the sensor electrodes superimposed on the graphical representation of a human head;

FIG. 15 is an exemplary graphical user interface for a BISx sensor, showing impedance status information, impedance values in ohms, and the location of the sensor electrodes superimposed on the graphical representation of a human head; and

FIG. 16 is an exemplary graphical user interface for a set of EEG sensors, showing impedance status information, impedance values in ohms, and the location of the sensor electrodes superimposed on the graphical representation of a human head.

### DETAILED DESCRIPTION

The following disclosure is presented to provide an illustration of the general principles of the present invention and is not meant to limit, in any way, the inventive concepts contained herein. Moreover, the particular features described in this section can be used in combination with the other described features in each of the multitude of possible permutations and combinations contained herein.

All terms defined herein should be afforded their broadest possible interpretation, including any implied meanings as dictated by a reading of the specification as well as any words that a person having skill in the art and/or a dictionary, treatise, or similar authority would assign particular meaning. Further, it should be noted that, as recited in the specification and in the claims appended hereto, the singular forms “a,” “an,” and “the” include the plural referents unless otherwise stated. Additionally, the terms “comprises” and “comprising” when used herein specify that certain features are present in that embodiment, but should not be interpreted to preclude the presence or addition of additional features, components, operations, and/or groups thereof.

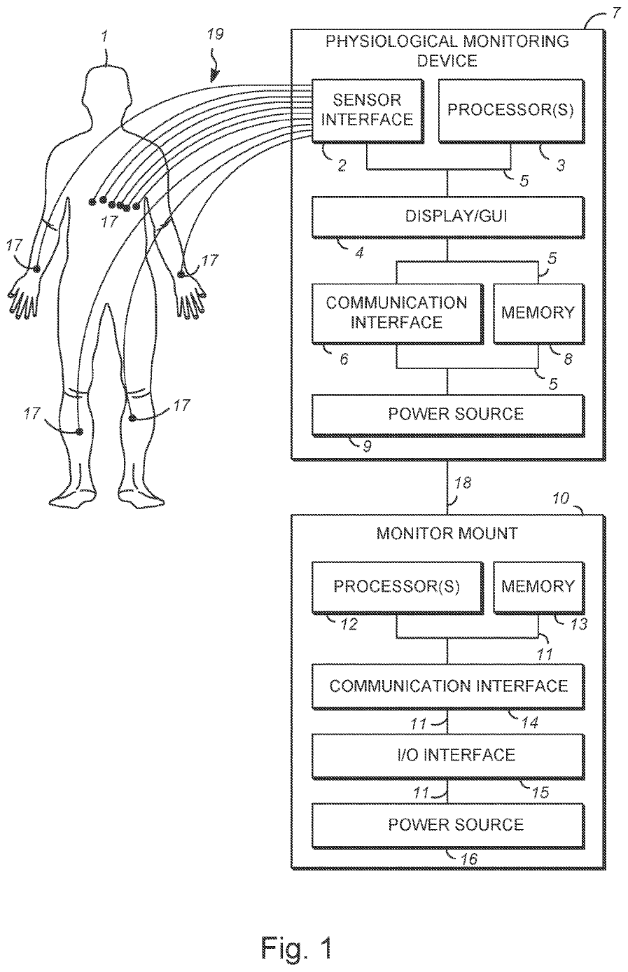

FIG. 1 is a schematic diagram of an exemplary physiological monitoring system 19 with a plurality of sensors 17 connected to a patient 1. For example, the plurality of sensors could include an ECG sensor having a plurality of surface ECG leads (electrodes) for detecting and analyzing ECG waveforms. As illustrated, the system includes a physiological monitoring device 7 capable of receiving physiological data from the sensors 17, and a monitor mount 10 to which the physiological monitoring device 7 can be removably mounted or docked.

The physiological monitoring device 7 is, for example, a patient monitor implemented to monitor various physiological parameters of the patient 1 via the sensors 17. The physiological monitoring device 7 includes a sensor interface 2, one or more processors 3, a display/GUI 4, a communications interface 6, a memory 8, and a power source 9. The sensor interface 2 can be implemented in software or hardware and used to connect via wired and/or wireless connections to one or more physiological sensors and/or medical devices 17 for gathering physiological data from the patient 1. The data signals from the sensors 17 include, for example, data related to an electrocardiogram (ECG), non-invasive peripheral oxygen saturation (SpO2), non-invasive blood pressure (NIBP), temperature, and/or end tidal carbon dioxide (etCO2), apnea detection, neuromuscular transmission (“NMT”), electroencephalogram (“EEG”), and brain monitoring external (“BISx”), and other similar physiological data.

The communications interface 6 allows the physiological monitoring device 7 to directly or indirectly (via, for example, the monitor mount 10) to communicate with one or more computing networks and devices (not shown). The communications interface 6 can include various network cards, interfaces or circuitry to enable wired and wireless communications with such computing networks and devices. The communications interface 6 can also be used to implement, for example, a Bluetooth connection, a cellular network connection, and/or a WiFi connection. Other wireless communication connections implemented using the communications interface 6 include wireless connections that operate in accordance with, but are not limited to, IEEE802.11 protocol, a Radio Frequency for Consumer Electronics (RF4CE) protocol, ZigBee protocol, and/or IEEE802.15.4 protocol.

Additionally, the communications interface 6 can enable direct (i.e., device-to-device) communications (e.g., messaging, signal exchange, etc.) such as from the monitor mount 10 to the physiological monitoring device 7 using, for example, a USB connection. The communications interface 6 can also enable direct device-to-device connection to other devices such as to a tablet, PC, or similar electronic device; or to an external storage device or memory.

The power source 9 can include a self-contained power source such as a battery pack and/or include an interface to be powered through an electrical outlet (either directly or by way of the monitor mount 10). The power source 9 can also be a rechargeable battery that can be detached allowing for replacement. In the case of a rechargeable battery, a small built-in back-up battery (or super capacitor) can be provided for continuous power to be provided to the physiological monitoring device 7 during battery replacement. Communication between the components of the physiological monitoring device 7 (e.g., 2, 3, 4, 6, 8, and 9) are established using an internal bus 5.

As shown in FIG. 1, the physiological monitoring device 7 is connected to the monitor mount 10 via a connection 18 that establishes a communication connection between, for example, the respective communications interfaces 6, 14 of the devices 7, 10. The connection 18 enables the monitor mount 10 to detachably secure the physiological monitoring device 7 to the monitor mount 10. In this regard, “detachably secure” means that the monitor mount 10 can secure the physiological monitoring device 7, but the physiological monitoring device 7 can be removed or undocked from the monitor mount 10 by a user when desired. The connection 18 may include, but is not limited to, a universal serial bus (USB) connection, parallel connection, a serial connection, coaxial connection, a High-Definition Multimedia Interface (HDMI) connection, or other similar connection known in the art connecting to electronic devices.

The monitor mount 10 includes one or more processors 12, a memory 13, a communications interface 14, an I/O interface 15, and a power source 16. The one or more processors 12 are used for controlling the general operations of the monitor mount 10. The memory 13 can be used to store any type of instructions associated with algorithms, processes, or operations for controlling the general functions and operations of the monitor mount 10.

The communications interface 14 allows the monitor mount 10 to communicate with one or more computing networks and devices (e.g., the physiological monitoring device 7). The communications interface 14 can include various network cards, interfaces or circuitry to enable wired and wireless communications with such computing networks and devices. The communications interface 14 can also be used to implement, for example, a Bluetooth connection, a cellular network connection, and a WiFi connection. Other wireless communication connections implemented using the communications interface 14 include wireless connections that operate in accordance with, but are not limited to, IEEE 802.11 protocol, a Radio Frequency For Consumer Electronics (RF4CE) protocol, ZigBee protocol, and/or IEEE 802.15.4 protocol.

The communications interface 14 can also enable direct (i.e., device-to-device) communications (e.g., messaging, signal exchange, etc.) such as from the monitor mount 10 to the physiological monitoring device 7 using, for example, a USB connection, coaxial connection, or other similar electrical connection. The communications interface 14 can enable direct (i.e., device-to-device) to other device such as to a tablet, PC, or similar electronic device; or to an external storage device or memory.

The I/O interface 15 can be an interface for enabling the transfer of information between monitor mount 10, one or more physiological monitoring devices 7, and external devices such as peripherals connected to the monitor mount 10 that need special communication links for interfacing with the one or more processors 12. The I/O interface 15 can be implemented to accommodate various connections to the monitor mount 10 that include, but is not limited to, a universal serial bus (USB) connection, parallel connection, a serial connection, coaxial connection, a High-Definition Multimedia Interface (HDMI) connection, or other known connection in the art connecting to external devices.

The power source 16 can include a self-contained power source such as a battery pack and/or include an interface to be powered through an electrical outlet (either directly or by way of the physiological monitoring device 7). The power source 16 can also be a rechargeable battery that can be detached allowing for replacement. Communication between the components of the monitor mount 10 (e.g., 12, 13, 14, 15 and 16) are established using an internal bus 11.

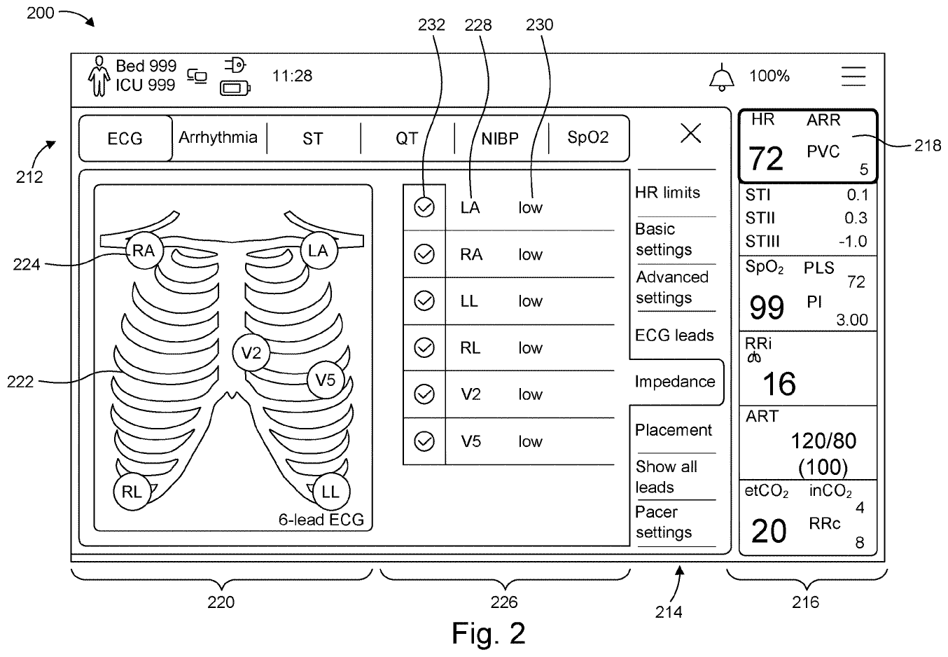

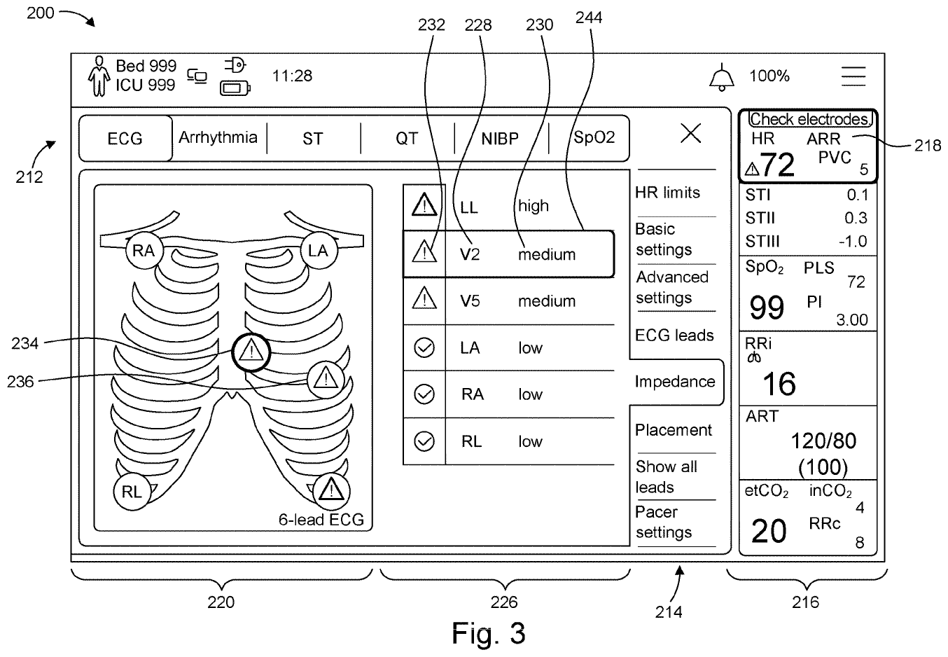

FIGS. 2 and 3 illustrate an exemplary graphical user interface (“GUI”) 200, which could be displayed on the display 4 of FIG. 1. The GUI 200 includes a horizontally-arranged sensor selection menu 212, which enables a user to select which if the available physiologic sensors is displayed. In FIGS. 2 and 3, an ECG sensor is displayed. The GUI 200 also includes a vertically-aligned view selection menu 214, which enables the user to select which view of the selected sensor will be displayed. The GUI 200 also includes a physiologic data window 216 in which physiologic data collected by the sensors connected to the sensor interface is displayed. Located at the top of the physiologic data window 216 physiologic data window 216 is a selected sensor data window 218 in which physiologic data and alerts relating to the sensor selected in the sensor selection menu 212 is displayed.

In FIG. 2, a six-lead ECG sensor is selected in the sensor selection menu 212 and an impedance view is selected in the view selection menu 214. When the impedance view is selected, the locations of electrodes of the ECG sensor are provided in a sensor location window 220, superimposed on a representation 222 of a human chest. The electrode locations are each identified by an icon 224 containing a label that identifies the electrode at that location (in this view, RA, LA, RL, LL, V2, and V5). In addition, a list containing the label 228, impedance status 230 and an impedance status icon 232 for each electrode is provided in a control window 226 that is located adjacent to the sensor location window 220.

In FIG. 2, the impedance status of all of the electrodes is “low”, which indicates that the impedance of each electrode is within the ideal impedance range for each electrode. The ideal impedance range represents an impedance range in which the electrode will provide accurate readings for the physiologic parameter being measured. The ideal impedance range could be provided by the manufacturer of the sensor, set by a user, or can be pre-determined by the physiological monitoring system 19 and will vary from sensor to sensor. Similarly, what constitutes a medium (greater than low impedance) or high impedance (greater than medium impedance) could be provided by the manufacturer of the sensor, set by a user, or can be pre-determined by the physiological monitoring system 19. Because each electrode is showing a low impedance status, the impedance status icon 232 (a check mark within a green circle) is intended to indicate that the electrode is operating properly. In addition, no warning message is visible in the selected sensor data window 218.

Referring to FIG. 3, the impedance status 230 of electrodes V2 and V5 is “medium”, which indicates that the impedance of each electrode is above the ideal impedance range for those electrodes, but is within operational limits. Because electrodes V2 and V5 are showing a medium impedance status 230, the impedance status icon 232 has changed to a caution icon (an exclamation point within a yellow circle), which is intended to indicate that the impedance of these electrodes could impact data accuracy. In addition, the a “check electrodes” alert is visible in the selected sensor data window 218. Finally, the label 234, 236 for electrodes V2 and V5 is replaced with the caution icon.

Similarly, the impedance status 230 of electrode LL is “high”, which indicates that the impedance of each electrode is outside of operational limits for that electrode, and therefore, needs attention. Because electrode LL is showing a high impedance status 230, the impedance status icon 232 has changed to a warning icon (an exclamation point within a red circle), which is intended to indicate that the impedance is likely to significantly impact data accuracy. In addition, the a “check electrodes” alert is visible in the selected sensor data window 218. Finally, the label 234, 236 for electrodes V2 and V5 is replaced with the warning icon.

Also shown in FIG. 3 is the ability for a user to select which one of the electrodes (in this case, electrode V2) from the sensor location window 220 is to be displayed in the selected sensor data window 218. Selection of an electrode is signified by a highlighted box 244 appearing around the list entry for that electrode in the view selection menu 214.

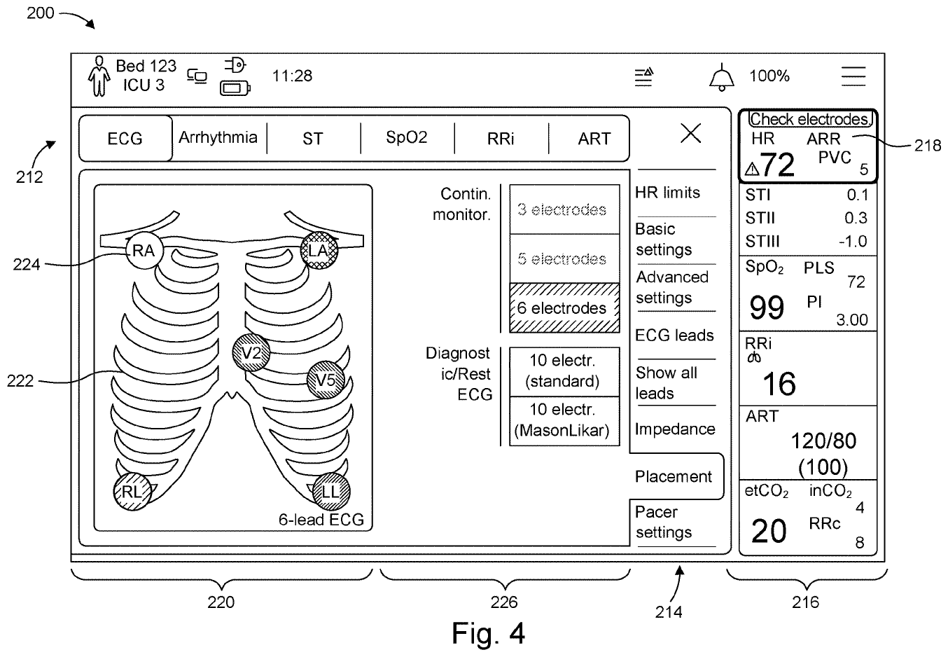

FIG. 4 shows the GUI 200 with the placement tab selected in the view selection menu 214. The placement tab is intended to assist clinicians in placing, identifying or confirming the proper location of each electrode of the sensor being displayed (in this case an ECG sensor) on the body of the patient. When the placement tab is selected, the locations of electrodes of the ECG sensor are provided in a sensor location window 220, superimposed on a representation 222 of a human chest. In this view, the icon 224 is a label that identifies the electrode that should be placed at that location on the body of the patient. In this view, no impedance-related icons are shown, regardless of the impedance status of each electrode. In this embodiment, the icons 224 are preferably color coded to match label colors on the actual electrodes. In the control window 226, a list of placement options is provided-in this example, ECG sensors having different numbers of electrodes, as well as different electrode placement options.

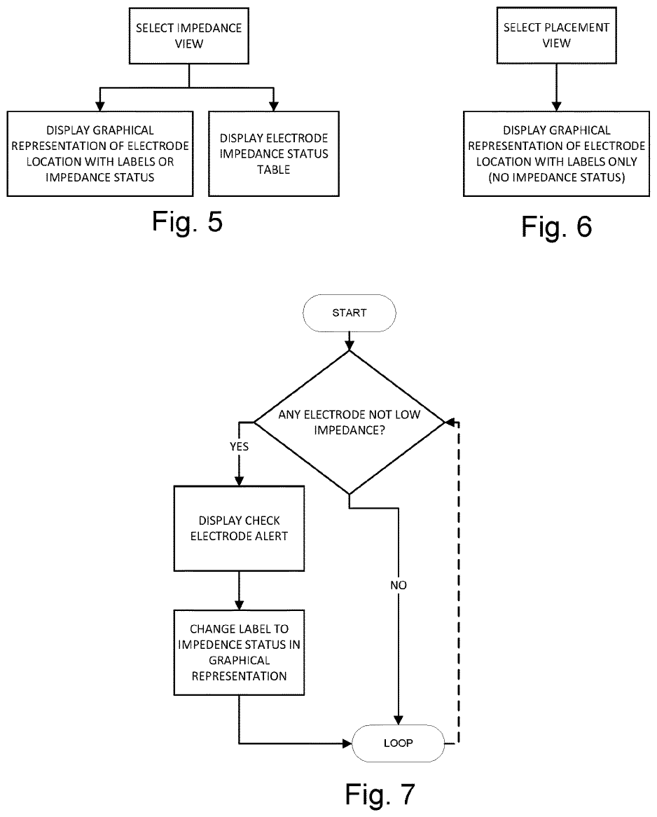

FIGS. 5 through 7 are flow charts showing exemplary steps used to operate the GUI to access impedance-related functionality. A general design principle is to show medium and high impedance indicators in areas of the display other than the control window 226 to draw attention to the need for clinical action. When impedance is low, impedance status information is only presented in the control window 226, in order to reduce visual clutter. One tap on the parameter shown on the main monitor provides high-level impedance information from which another tap provides the detailed electrode location and impedance status in combination.

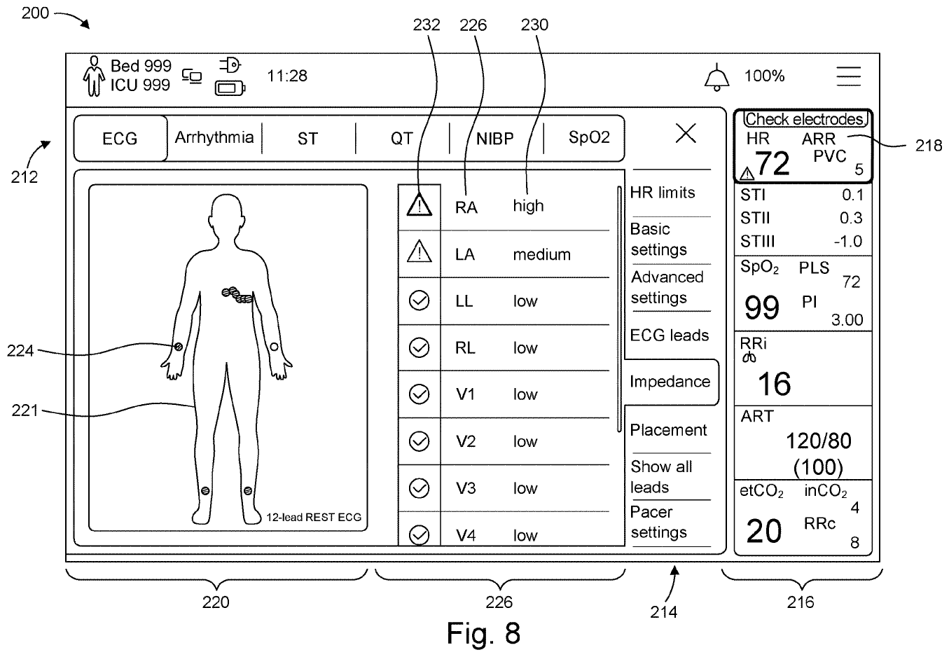

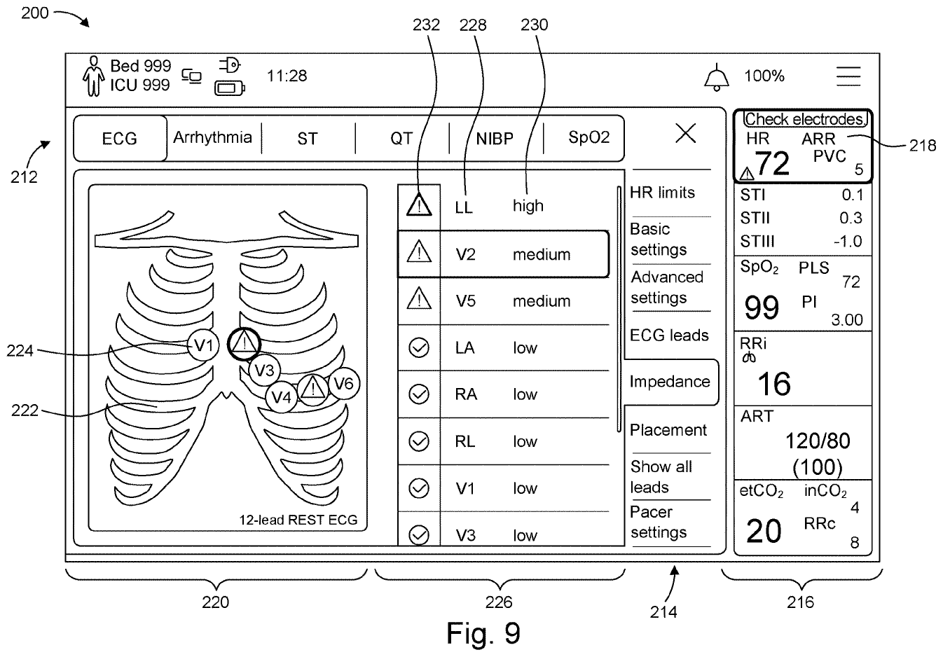

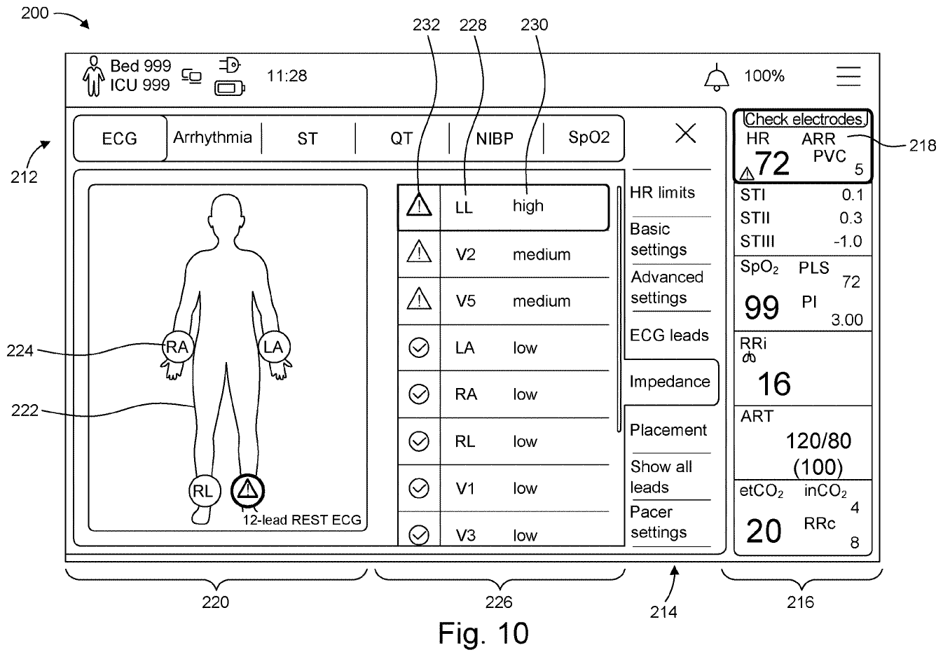

FIGS. 8 through 10 show an exemplary GUI 200 for a twelve-lead ECG sensor. Due to the higher number of electrodes, this embodiment includes multiple user-selectable views each showing a different graphical representation of the human body. In FIG. 8, a full body view is shown in the sensor location window 220. In FIG. 9, a chest view is shown in the sensor location window 220. In FIG. 10, a limbs only view is shown in the sensor location window 220. As can be seen in FIG. 8, the icons 224 located on the graphical representation of the human body may be smaller than the icons shown in the chest view (FIG. 9). These different views enable the clinician to have more complete information concerning the location and impedance status of the electrodes.

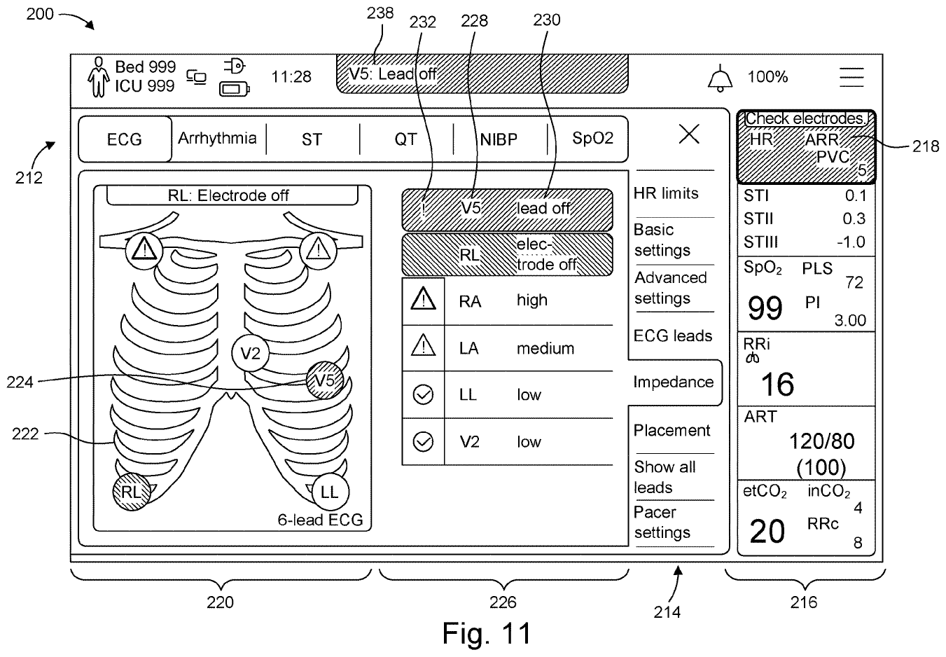

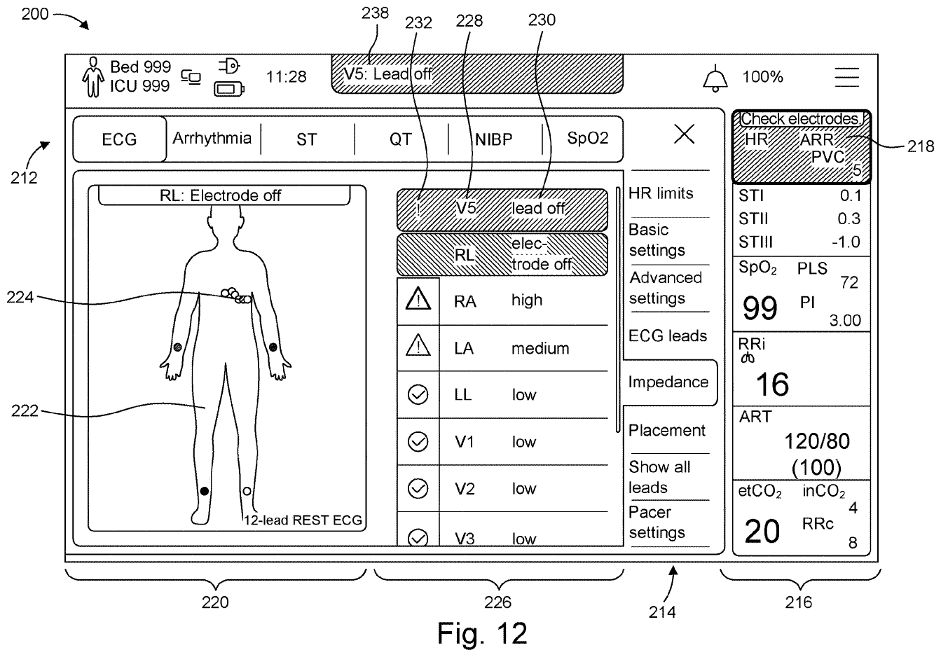

FIGS. 11 and 12 show the GUI 200 of FIGS. 2 and 8, respectively, with “lead off” and “electrode off” warnings being displayed for electrodes V5 and RL, respectively. These warnings are intended to indicate that an electrode lead is disconnected and needs attention. Because the status of electrode V5 is “lead off”, the area in which electrode V5 is listed in the control window 226 is filled with a different color (in this example, blue) than the other areas on the control window 226. In addition, the icon 224 for electrode V5 in the sensor location window 220 is also preferably filled with the same color (blue) as the area in which electrode V5 is listed in the control window 226. In addition, the selected sensor data window 218 is preferably filled with the same color as area in which electrode V5 is listed in the control window 226. Finally, because a disconnected electrode lead will result in inaccurate physiologic data, an additional alert 238 is provide along the top of the GUI 200 for the purpose of drawing the clinician's attention to the issue. Similar changes in the GUI 200 are provided for the “electrode off” warning, except that the fill color is white instead of blue.

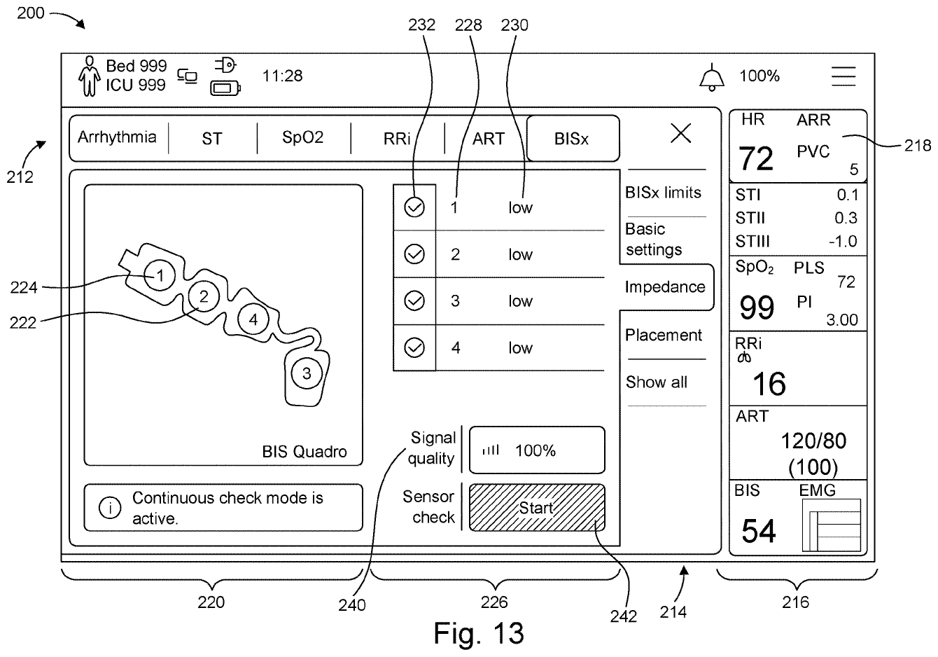

FIG. 13 shows the GUI 200 with a BISx sensor selected in the sensor selection menu 212. In this exemplary embodiment, the representation 222 in the sensor location window 220 is a representation of an electrode retaining device instead of a representation of portion the human body. In addition, the control window 226 includes a user-selectable button 242 to initiate a sensor check. The user-initiated sensor check is intended to enable a clinician to obtain electrode impedance information for sensors (or specific electrodes on a sensor) for which real-time impedance readings are not possible. For example, in some embodiments it may be challenging to obtain electrode-level impedance status for EEG electrodes while simultaneously providing the patient data collected by those electrodes. In other words, for some sensors and in some embodiments, electrode impedance status can only be measured by interrupting the flow of patient data collected by that sensor. Accordingly, providing a user-initiated electrode-level impedance status check enables the clinician to decide whether and when it is necessary to obtain impedance information and interrupt the flow of patient information for that sensor.

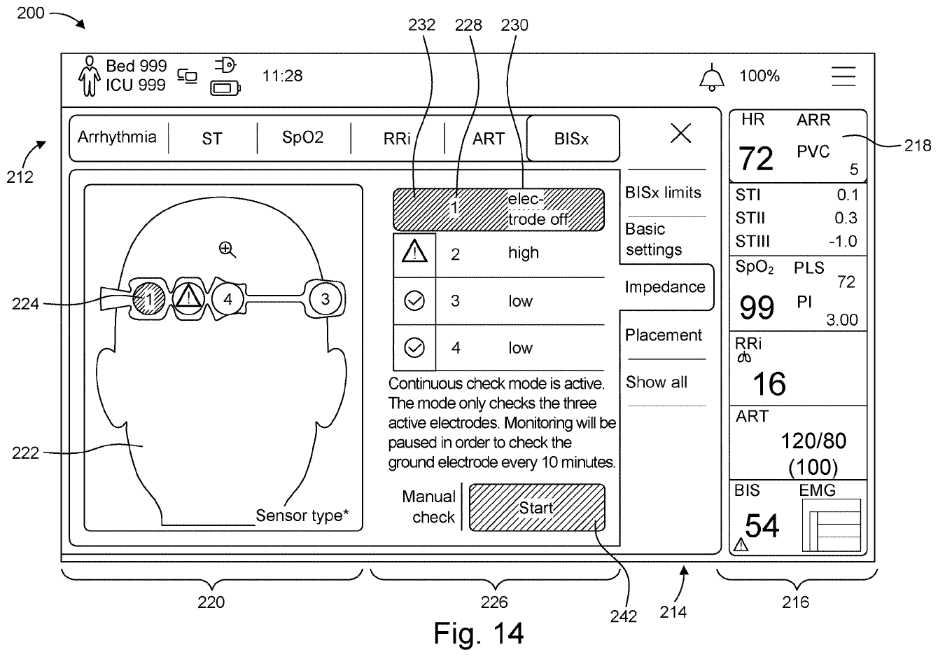

FIG. 14 shows another GUI 200 with a BISx sensor selected in the sensor selection menu 212. In this exemplary embodiment, the representation 222 in the sensor location window 220 is a representation of a human head.

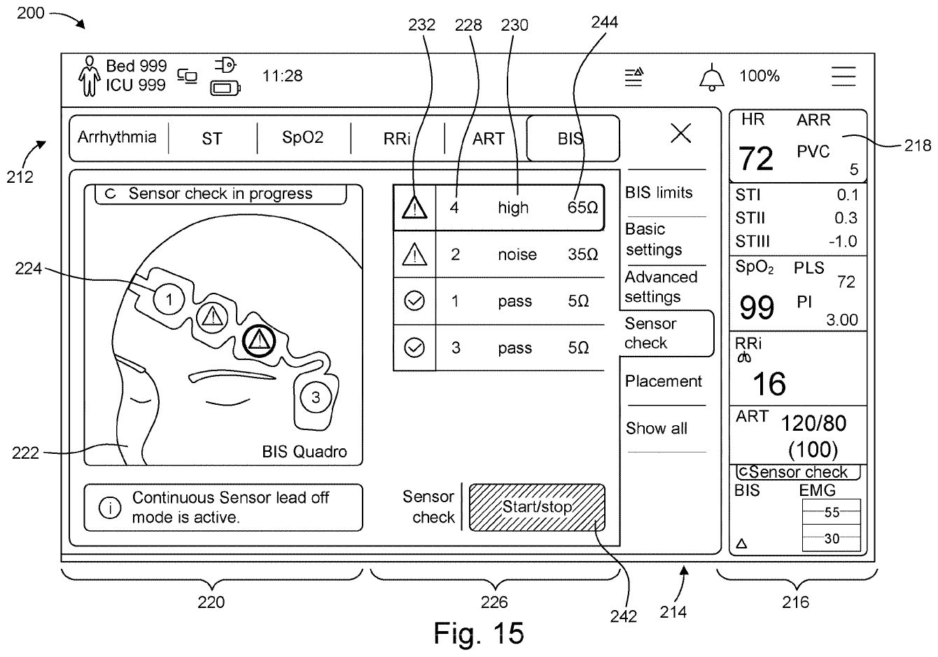

FIG. 15 shows graphical user interface 200 for a BISx sensor, showing impedance status information 230, impedance values in ohms 244, and the location of the sensor electrodes superimposed on the graphical representation of a human head 222. In FIG. 15, the impedance status 230 of some electrodes 224 is indicated as “pass” with an impedance of 5 ohms. A low impedance, or pass impedance, indicates that the impedance of that particulate electrode is within the ideal impedance range. The ideal impedance range represents an impedance range in which the electrode will provide accurate readings for the physiologic parameter being measured. The ideal impedance range could be provided by the manufacturer of the sensor, set by a user, or can be pre-determined by the physiological monitoring system 19 (FIG. 1) and will vary from sensor to sensor. In some embodiments, a low impedance or pass impedance is less than 10 ohms, less than 8 ohms, less than 5 ohms, or less than 4 ohms. Some electrodes in FIG. 15 are indicated as being medium impedance status 230. In some embodiments, medium impedance is between 40 ohms and 5 ohms, between 35 ohms and 10 ohms, between 40 ohms and 10 ohms, or between 35 ohms and 5 ohms. In some embodiments, high impedance is greater than 15 ohms, greater than 18 ohms, or greater than 20 ohms. Each electrode 224 having a pass or low impedance status 230 is also indicated with a check mark within a green circle as an impedance status icon 232 to indicate that the electrode 224 is operating properly. Different impedance status icons 232 are visible in the selected sensor data window to indicate either medium impedance status, high impedance status, or noise. In some instances, the impedance status icon may be a triangle with an exclamation point within the triangle 232. Optionally, the impedance status icon 232 for a high impedance status could be differentiated from medium impedance status (e.g. having a heavier line weight, different color, etc.).

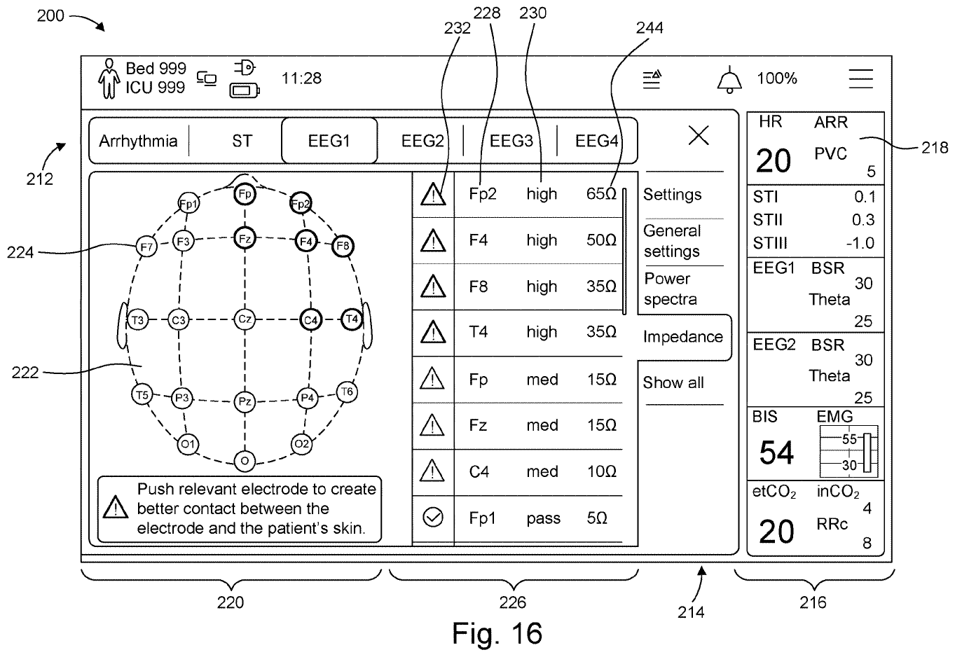

FIG. 16 shows a graphical user interface 200 for a set of EEG sensors 224, showing impedance status information 230, impedance values 224 ohms, and the location of the sensor electrodes superimposed on the graphical representation of a human head 222. Similar ranges may be provided for high impedance status, medium impedance status, low impedance status as shown in FIG. 15. Similarly, warning messages 232 may be provided with the impedance reading is outside the low or pass range.

All examples and conditional language recited herein are intended for pedagogical purposes to aid the reader in understanding the principles of the present invention and the concepts contributed by the inventor in furthering the art. As such, they are to be construed as being without limitation to such specifically recited examples and conditions. Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

It is to be understood that the embodiments described herein are merely exemplary and that a person skilled in the art may make many variations and modifications without departing from the spirit and scope of the invention. All such variations and modifications are intended to be included within the scope of the invention, as defined by the following claims.

1. A method comprising:
(a) storing electrode data associated with each of at least one type of electrode-based sensor, each of the at least one type of electrode-based sensor having a plurality of electrodes, the electrode data comprising (1) a label for each of each of the plurality of electrodes, (2) a location of each of each of the plurality of electrodes on a representation of at least a portion of a human body or an electrode-retaining device, and (3) a plurality of impedance statuses comprising a low impedance status and a high impedance status, the electrode data being accessible by a patient monitoring system having a display;
(b) selecting one of the at least one type of electrode-based sensor;
(c) for the at least one type of electrode-based sensor detected in step (b), providing on the display a graphical representation of the location for each of the plurality of electrodes stored in step (a);
(d) receiving impedance data for each of the plurality of electrodes of the at least one type of electrode-based sensor detected in step (b); and
(e) displaying on the display the label and an applicable impedance status for each of the plurality of electrodes as a function of the plurality of impedance statuses stored in step (a) and the impedance data received in step (d).

2. The method of claim 1, further comprising:
(f) displaying an electrode failure alert if any of the applicable impedance status displayed for any of the in step (e) comprises an impedance status other than the low impedance status.

3. The method of claim 1, further comprising:
(g) displaying patient physiologic data gathered the at least one type of electrode-based sensor selected in step (b) in a physiologic data window in the display.

4. The method of claim 3, further comprising:
(h) displaying in the physiologic data window patient physiologic data gathered by at least one sensor other than the at least one type of electrode-based sensor selected in step (b).

5. The method of claim 1, wherein the label and an applicable impedance status for each of the plurality of electrodes displayed pursuant to step (e) is displayed in the form of a table.

6. The method of claim 5, wherein the table is positioned adjacent to the graphical representation of step (c).

7. The method of claim 1, wherein step (a) further comprises associating an icon with each of plurality of impedance statuses.

8. The method of claim 7, further comprising displaying the icon associated with the applicable impedance status for each of the plurality of electrodes determined in step (d) superimposed on the location of the electrode displayed in the graphical representation of step (c).

9. The method of claim 1, further comprising:
(i) displaying the label and location of the electrode displayed in the graphical representation of step (c) without an impedance status.

10. The method of claim 1, wherein the graphical representation displayed in step (c) comprises the representation of the at least a portion of a human body or the electrode-retaining device stored in step (a).

11. The method of claim 1, wherein the graphical representation displayed in step (c) comprises the representation of the at least a portion of a human body.

12. The method of claim 11, wherein the graphical representation displayed in step (c) comprises a chest view and a full body view.

13. The method of claim 12, wherein the graphical representation displayed in step (c) is user selectable.

14. The method of claim 1, wherein the plurality of impedance statuses further comprises a medium impedance status.

15. The method of claim 1, wherein step (b) comprises selecting one of the at least one type of electrode-based sensor based on user input to the display.

16. The method of claim 1, wherein step (b) comprises selecting one of the at least one type of electrode-based sensor by detecting a connection between the selected one of the at least one type of electrode-based sensor and a sensor interface of the patient monitoring system.

17. The method of claim 1, wherein the patient monitoring system is capable of performing steps (c) and (e) for each of the at least one type of electrode-based sensor.

18. The method of claim 17, wherein the at least one type of electrode-based sensor comprises an ECG sensor and a BISx sensor.

19. The method of claim 18, wherein the at least one type of electrode-based sensor further comprises an EEG sensor and a NMT sensor.

20. A system for analyzing and displaying patient health information, the system comprising:
at least one physiological sensor configured to obtain health information from a patient via a plurality of electrodes;
a memory configured to store health information from the at least one physiological sensor;
an electronic display having a user interface for receiving commands from a user; and
a processor being in electrical communication with the at least one physiological sensor through a sensor interface, the memory, and the electronic display, the processor being configured to perform the steps of claim 1.
